(19) United States
(12) Patent Application Publication
(10) Pub. No.: US 2024/0045004 A1
(43) Pub. Date: Feb. 8, 2024

(54) MRI Compatible Projector Assembly and System with Collimated Optics Through an RF Waveguide for In-Bore Viewing

(71) Applicant: PDC Facilities, Inc., Hartland, WI (US)

(72) Inventors: Matthew K. Murphy, Mukwonago, WI (US); James P. Maslowski, Wauwatosa, WI (US)

(21) Appl. No.: 18/364,636

(22) Filed: Aug. 3, 2023

Related U.S. Application Data
(60) Provisional application No. 63/395,198, filed on Aug. 4, 2022.

(51) Int. Cl. G01R 33/28; A61B 5/055

(52) U.S. Cl. CPC G01R 33/283; A61B 5/055

(57) ABSTRACT

Systems and methods for projecting an image onto an interior surface of an MRI machine includes projection through a waveguide and image adjustment. The projector is generally placed within an RF-protective box and spaced from the MRI machine. The projector emits a light and image projection through the waveguide, which travels into a fold minor lens located within the MRI bore, which then redirects the image onto the inside of the bore.

### RELATED APPLICATIONS

This application claims the benefit of U.S. Provisional Patent Application Ser. No. 63/395,198, filed 4 Aug. 2022, and entitled “MRI Compatible Projector Assembly and System with Collimated Optics through an RF Waveguide for In-Bore Viewing,” which is incorporated herein by reference in its entirety.

### BACKGROUND OF THE INVENTION

The reality of a trip to the hospital is that most patients would rather be somewhere else. This is particularly true of patients who are ordered to take a magnetic resonance imaging (MRI) scan by their physician. The MRI tube where the patient is enclosed can further elevate already-increased stress levels and add an element of claustrophobia. The anxiety-heightening experience of being enclosed in a tight, loud tube has the potential to alienate patients, preventing them from receiving the healthcare they need to recover. A way to attempt to relax the patient is to create a viewable image as a distraction for the patient undergoing the MRI, typically in the form of a static ceiling image in the medical imaging room. However, there is a need for an improved assembly and system that is novel, constituting a major improvement to over the prior art.

This invention relates generally to medical diagnostic medical imaging rooms and to medical diagnostic imaging equipment, such as magnetic resonance imaging (or “MRI”) machines or scanners, such machines being configured for placement of a patient within a bore (or “magnet bore”) that is surrounded by a superconducting electromagnetic coil that generates a strong static magnetic field for medical diagnostics. Such machines are well known in the art. This invention also relates generally to devices that are ancillary to such machines and that are used for the purpose of relieving patient anxiety when a patient is placed within the bore of an MRI machine. More specifically, the present invention relates to an MRI-compatible projector and projection system that uses collimated optics through a radio frequency (or “RF”) waveguide and a fold minor lens (or “FML”) to create an image within the MRI bore for in-bore viewing by the patient.

In the experience of these inventors, there are a number of ways to create an image that is viewable by a patient who is disposed within the bore of an MRI machine (also referred to herein as an “in-bore” image), such bores being relatively small and claustrophobic for most patients. One common way to relax the patient is to create a viewable image as a distraction for the patient. This image is typically a static ceiling image used in the medical diagnostic medical imaging room. However, these inventors believe that there is a need for an improved assembly and system that is novel and which constitutes a major improvement over the prior art.

### SUMMARY OF THE INVENTION

Embodiments of systems and methods according to the present invention relate generally to an assembly and system that uses a video projector inside an MRI scan room whereby a projected image can be displayed on an inner surface of the magnet bore. Alternatively, the projected image could be displayed on a diffuser placed at an end of the MRI scanner. It is desired to have the projector disposed outside the strong static magnetic fields of the scanner, enclosed in an RF housing, and to obtain very low radiated and conducted emissions by sending the projection beam through an RF waveguide.

According to an aspect of an embodiment of an MRI-compatible projector system according to the present invention, it includes a radiofrequency-shielded box at least substantially enclosing an image projector, the projector comprising a lens and configured to output a light and image projection. The system further includes a waveguide, which may be cylindrical in cross-section and/or an extension of the RF box, configured to receive an image from the projector lens, the waveguide also comprising a plurality of secondary optics elements. The system further includes an image redirection module in the form of a fold-mirror lens (FML) spaced from the waveguide and configured to receive the light and image projection of the projector, having passed through the secondary optics, and reflect the projection onto the inner surface of the MRI machine bore to form an image.

According to another aspect of embodiments of an MRI-compatible projector system according to the present invention, the projector may be a digital light processing (DLP) projector, a liquid crystal on silicon (LCoS) projector, a laser projector, or other image projecting device capable of spaced projection of an image onto an at least partially reflective surface to be perceived by a human eye.

According to still another aspect of embodiments of an MRI-compatible projector system according to the present invention, at least one of the plurality of secondary optics elements may include an aspherical lens, an achromatic lens, a doublet lens, or a plano-convex lens, or other optics elements (which may be fixed or adjustable) capable of forming or transmitting a collimated beam.

According to yet another aspect of an embodiment of an MRI-compatible projector system according to the present invention, the focal length or distance of the projector is adjustable.

According to a further aspect of an embodiment of an MRI-compatible projector system according to the present invention, the system may further include a sensor configured to detect a position of an image receiving support (e.g., and MRI patient table within the MRI machine bore) or reflector/transmitter (e.g., FML) and transfer data related to the position to the projector. The focal length or distance of the projector may be adjusted automatically, according to the position, to allow transmission of a predetermined amount or portion (e.g., a majority) of the projected image onto the reflector/transmitter (e.g., FML). Additionally or alternatively, the secondary optic may be adjusted automatically according to the position.

According to still a further aspect of an embodiment of an MRI-compatible projector system according to the present invention, the reflector/transmitter (e.g., FML) is positioned at a distance of between about five and about nine feet, more preferably at approximately seven feet, from a nearest portion of the waveguide.

According to an aspect of another embodiment of an MRI-compatible projector system according to the present invention, it includes a radiofrequency-shielded projector, the projector comprising a lens and configured to output a light and image projection. The system further includes a waveguide, which may be cylindrical in cross-section and/or an extension of the RF box, configured to receive an image from the projector lens, the waveguide also comprising a plurality of secondary optics elements. At least one secondary optics element comprises a fisheye lens configured to project the light and image projection onto an inner surface of an MRI machine bore.

According to an aspect of a method according to the present invention, the method includes the step of providing a projection system according to the present invention, placing the projector in a spaced relationship from a reflector or transmitter, or support therefor, at a distance of about, but more preferably at least, five feet, and placing the reflector or transmitter (e.g., FML) at least five feet from a nearest portion of the waveguide, which such placement may be in an MRI machine bore. A light and image is projected through the waveguide and to the reflector/transmitter, which is then reflected onto a display surface, which may be an inner surface of the MRI machine bore.

According to another aspect of a method according to the present invention, the focal length or distance of the projector may be manually or automatically adjusted in response to placement and/or movement of the reflector or transmitter, or support therefor (e.g., movement of a patient table within an MRI bore).

### BRIEF DESCRIPTION OF THE DRAWINGS

FIG. 1 is a representative illustration of the system showing the projector, RF enclosure, waveguide with optics, prism, lens assembly, and the light and image projection outputs created by this system.

FIG. 2 is side elevational of the system and showing the projector and FML for a head-first scan in maximum position entering the scanner.

FIG. 3 is a view similar to that shown in FIG. 2 but showing the projector and FML for a head-first scan in maximum position exiting the scanner.

FIG. 4 is an enlarged view of a portion of FIG. 3 and showing the system in greater detail.

FIG. 5 comprises multiple views of the system in accordance with the present invention.

### DESCRIPTION OF THE PREFERRED EMBODIMENT

Although the disclosure hereof is detailed and exact to enable those skilled in the art to practice the invention, the physical embodiments herein disclosed merely exemplify the invention which may be embodied in other specific structures. While the preferred embodiment has been described, the details may be changed without departing from the invention, which is defined by the claims.

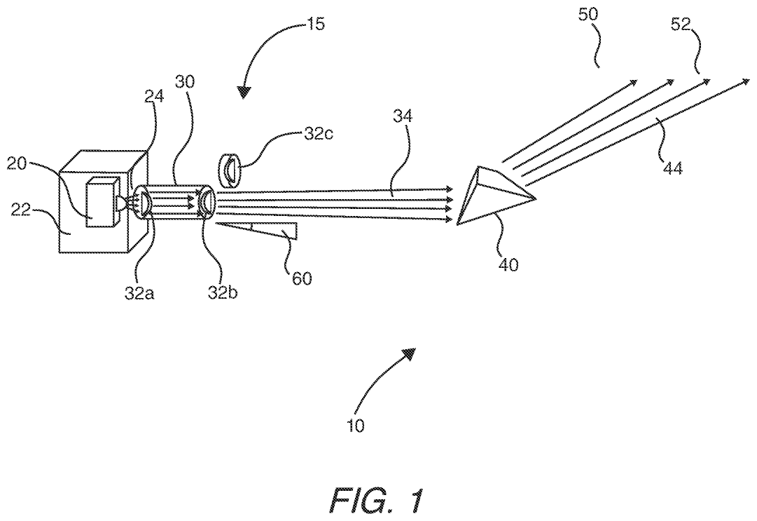

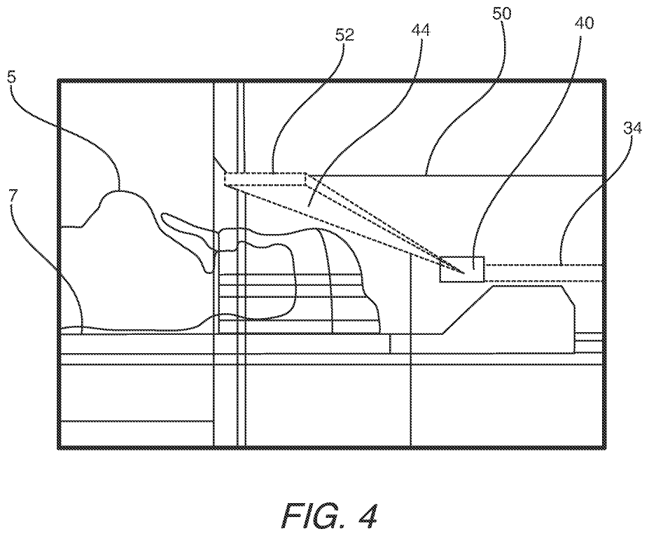

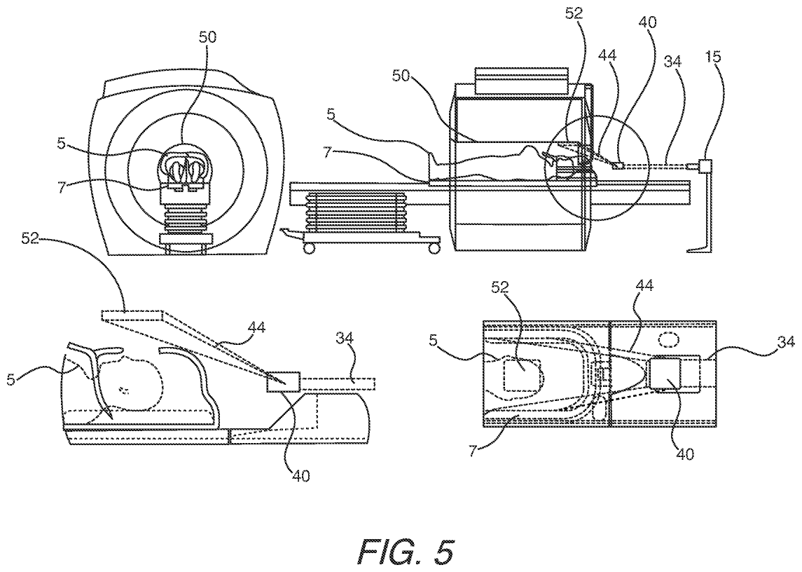

Turning now to FIGS. 1-5, a first embodiment 10 of a projection system according to the present invention can be seen.

The present invention is based on a desire to have a video projector disposed inside an MRI scan room with the image generated by the projector displayed on the inner ceiling of the magnet bore. Alternatively, the projected image could be displayed on a diffuser placed at the back of the MRI scanner. It is also desired to have the projector located outside the strong static magnetic fields of the scanner, the projector being enclosed in an RF housing so as to obtain very low radiated and conducted emissions by sending the projection through an RF waveguide. These requirements dictate that a projector with a large “throw ratio” be developed, the throw ration being the distance from the lens to the screen (i.e., the “throw”) to the screen width (i.e., the ratio of distance/width).

Further, there is not much room inside the MRI bore which means that the image needs to be relatively small. Most projectors have a small throw ratio. In accordance with the present invention, and having the projector disposed outside the bore, at some distance, will require a large throw ratio. Put another way, a larger throw ratio corresponds to a more tightly focused optical system.

Also, almost all cameras, windows, and displays within a scan room require a conductive mesh for electromagnetic interference (or “EMI”) attenuation in front of the viewing area. In such a system, the wire EMI mesh obstructs and degrades the image.

Based on its geometry, an RF waveguide can also attenuate EMI and does not obstruct the image (i.e., requiring no RF mesh to attenuate EMI). Using a collimated projector beam, through a waveguide with no mesh, is the key to increasing the image quality. Glass, acrylic, or a non-electrically conductive optic element can be placed in the RF waveguide cylinder and be used to create a lens system that increases the throw ratio, collimates the beam, and allows for unobstructed image quality.

The projector can be any type of projector including, but not limited to, digital light processing (or “OLP”), liquid crystal on silicon (or “LCoS”), or laser. The projector could be a small hand-held pico-projector or a large conference room style projector of typical construction. Using additional mirror, prisms, or lens the projector can be oriented, and mounted, to meet unique design specifications.

Referring now to the drawings in detail, FIG. 1 illustrates a schematic showing the assembly and system, generally identified 10, configured in accordance with the present invention. The assembly and system 10 further comprises a projector subassembly, generally identified 15, wherein an image projector 20 is enclosed within an RF box 22 with a light and image projection output 24 from the projector 20 that is coupled to certain collimating/focusing optics contained within or at the output of an RF waveguide 30. The optics could be as many segments as are required to meet performance parameters. The secondary optics 32a, 32b, 32c could include an aspherical, achromatic, doublet, piano-convex lens, etc. which perform the beam collimation, image focus, and other functions. However, multiple stages and other types of lenses could be used to compensate for chromatic aberration, spherical aberration, tighter divergence, and other optical aberrations. The beam collimation is required to allow the light and image projection 34 to traverse the RF waveguide 30 without having the light and image projection cut off and keep the image sized properly for a receiving surface of the FML 40.

The secondary optics 32a, 32b, 32c, for example, are housed within or just outside the cylindrical RF waveguide 30 which has dimensions allowing for suppression of electromagnetic radiation from 20 MHz to 300 MHz. Typically, a tube length to diameter ratio of 4 is sufficient. If the material in the waveguide 30 is not electrically conductive the waveguide 30 will effectively suppress the radiation. The waveguide 32 serves as a housing for the optics as well.

When the light and image projection 34 leaves the waveguide 30 it must travel a variable distance of 5 to 9 feet, be in focus, remain constant size, and land on a minor or prism surface of the FML 40. Therefore, the throw ratio of the system 10 must be large and variable. To complicate matters, the distance from projector to FML 40 will be variable from 5 feet to 9 feet or greater when a patient 5 is moved inside the MRI scanner bore 50. The projector 20 could be placed at various distances with respect to the back of the bore. Therefore, some type of active feedback with respect to distance from projector 20 to FML 40 is required. The optics could be actively modified to account for the increase/decrease in distance and the light and image projection 44 from the FML that is used to create the projected image 52 can be resized and fit to the static 3″×2″ target. However, it might be acceptable to split the difference and design for a 7 foot distance. The result of splitting the difference would be having the image smaller at 5 feet (possibly some image cut off due to the aperture of the FML 40) and larger at 9 feet.

In short, the light and image projection 34 produced by the projector 20 and the secondary optics is projected into the MRI machine and received by the FML 40 which is located behind the patient 5's head. The FML 40 will serve to fold, throw forward, and expand the in-bore light and image projection 44 to form an image 52 on the inner ceiling 50 of the MRI machine. This image 52 will be displayed in front of the patient 5. This solution offers the patient 5 in-bore viewing with no obstructions for viewing external video displays (minor, glasses, display, etc.).

An additional embodiment would be the projector system described above without the folding, and expanding, optic in the bore (i.e., the FML). A “fisheye lens” (also known as also known as an “ultra wide” or “super wide” lens which can capture an extremely wide image, typically around 180°) is placed outside the RF waveguide 30 and the projector/fisheye system would be placed behind the bore and the image 52 would be projected onto all surfaces of the inner magnet wall 50. This would create a type of three-dimensional view inside the bore with all surfaces having a projected image, thereby giving the image a dynamic feel for the patient 5.

Yet another embodiment is to have the projector, and system, using 3D effects to make the bore disappear. A change in the focal point to a couple meters away could help a patient 5 with a stigmatism view the image clearly without needing his or her glasses. By shifting the focal point, a virtual image is created. This embodiment would use some type of holographic film, multi-perspective video encoding, and tight full width half max (or “FWHM”) laser projector. These inventors envision incorporating some type of heads up display (or “HUD”) technology with augmented reality or virtual reality (or “ARNR”) computer-generated simulations.

Another variant possible is an additional optic used to increase the angle of divergence 60. If a scan is performed with a coil having its own mirror, it might be desirable to project the image on a semi-transparent diffuser (not shown) located at the back of the magnet bore opening. Displaying an image on a screen behind the bore is not unique but the additional optic, at the output of the RF waveguide 30, to increase divergence angle 60 of the collimated beam is. By increasing the divergence angle 60 the projected image will greatly increase in size and the throw ratio decreased when a diffuser/screen is placed closer to the projector 20.

It is to be understood that all dimensions stated are quite typical but bore size, distance, target size, etc. tend to change as designs are changed. The system 10 described would only need to be recalculated and adopted for new geometries. Versavideo® (a registered mark of PDC Facilities, Inc.) software and hardware will allow for flipping image, slicing video to display only desired content, Up/Down converting resolution, insert overlays, correct for image keystone created by the bore, and keep the image a constant size.

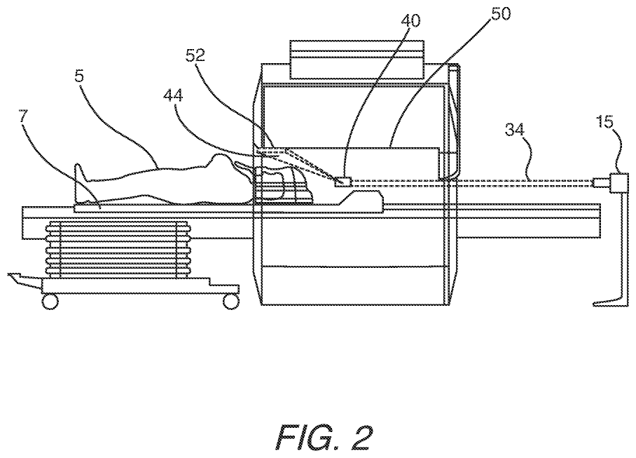

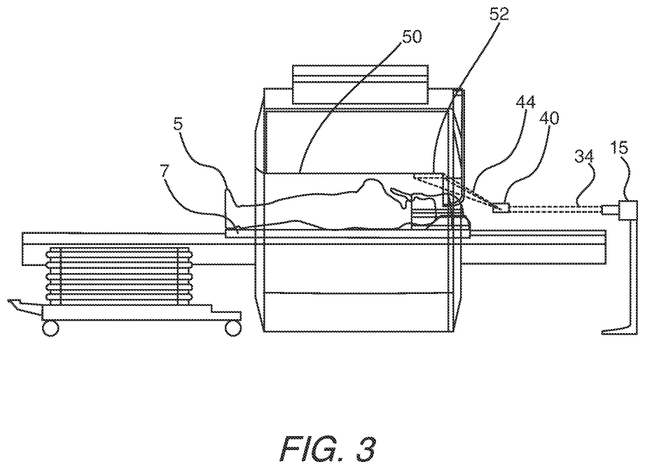

The essence of the present invention can include the following specific constructs (without specific reference to element numbers shown in FIG. 1):
1. Locating a projector in an RF box and placing it some distance behind the bore (greater than 5 feet). This location will keep the electronics out of high static magnetic field, be electrically quiet with respect to EMI, and eliminate the need to add equipment into an already crowded scanner bore.
2. The projector could be located either in front of the scanner or behind the scanner depending upon head first or feet first scan. The projector could be mounted on a pedestal or on the wall inside the scan room. The orientation of the projector could be horizontal or vertical and any rotation including 90°, 180°, 270°, or 360°.
3. Directing the light and image projection through a waveguide on the RF enclosure. This solves two problems:
   a. Eliminates the need for an RF mesh in front of the projector and will not degrade the light and image projection.
   b. The secondary projection optics required to send the light beam through a waveguide will naturally collimate the light and image projection and can be designed for a desired or custom throw ratio.
4. Having a FML inside the bore to translate the light and image projection and make the image appear on the top of the bore inside the scanner. It is estimated that the size of the FML will be 5″×5″×3″. The FML has no active electronics and is made of non-ferrous low hydrogen content material, which minimizes interference with the scan.
5. The ability to adjust the optical system focal length when the patient table 7 moves within the bore. See FIGS. 3-5. This will keep the image in focus as the table moves in the bore. In the present embodiment this is accomplished by moving the projector with respect to the lens elements. One could design the lens with a focus adjust and leave the projector in place.
6. The ability to adjust the collimating optic so the angle of divergence changes when the patient table 7 moves within the bore. This would ultimately keep the image size on the FML constant as the table moves in the bore. This can also be accomplished with a digital zoom technique using an ASIC (a unique type of integrated circuit meat for a specific application), FPGA (a reprogrammable integrated circuit), Versavideo® software and hardware, etc.
7. Track the patient table 7 location. This could be direct feedback for the magnet manufacturer, encoders, laser measurements, etc.
8. Given the long throw ratio, the projector can be lower lumen because all light will be “collected” and displayed in a relatively small area. The projector could be LCoS, OLP, laser, or any other type of projector, as previously mentioned.
9. Use either optics or digital processing to correct for image distortion/keystone due to curvature of the bore.
10. Add a third divergent optic element, outside the RF enclosure/waveguide, which decreases the throw ratio so the beam will diverge faster and can be displayed on a diffuser/screen behind, and outside, the bore. This might be needed if a scan uses a coil which already has a mirror. The proposed lens would tilt into place in front of the RF waveguide when needed. The patient 5 can view the image using prism glasses, mirror glasses, coil mirror, etc. This technique could also be useful if the patient 5 orientation is changed (e.g., feet first entry into MRI scanner).
11. As alluded to above, the system can be configured as feet first scan or head first scan. In feet first the projector would be in front of the scanner and the FML would be located on the front side of the patient table 7. In a head first scan the projector will be located behind the scanner and the FML would be located on the back side of the patient table 7. Again, see FIGS. 2-4.
12. The size of the image could vary for patient 5 preference, type of scan, functional MRI, etc. That is, the image size will remain constant throughout travel but can be of various size dependent on the type of scan.

In summary, the salient features of the present invention are that the projector is outside the bore, away from the high static magnetic field, and the image is sized for viewing in the bore. Image quality will be optimal because no mesh is required for EMI attenuation. The image will display on the ceiling of the bore allowing maximum separation from the patient 5's eye. Other designs propose a viewing device between the patient 5 in the scanner and the top of the bore. Cost will be low because of a simple RF enclosure, off the shelf optics, and low power projector. Most all projectors are designed for small throw ratios (large image close to the projector) and must be modified for the larger throw ratio (small image far away from the projector). Some solutions have been a monitor behind the bore with a mirror for the patient 5 to view. One example of a monitor is an LCD in a RF enclosure with RF mesh over the screen.

The foregoing is considered as illustrative only of the principles of the invention. Furthermore, because numerous modifications and changes will readily occur to those skilled in the art, it is not desired to limit the invention to the exact construction and operation shown and described. While the preferred embodiment has been described, the details may be changed without departing from the invention, which is defined by the claims.

What is claimed is:

1. An MRI-compatible projector system comprising:
an RF box enclosing a projector, the projector comprising a lens and configured to output a light and image projection;
a cylindrical waveguide configured to be in mechanical communication with the projector lens, the waveguide also comprising a plurality of secondary optics; and
a fold-mirror lens (FML) disposed within an MR machine bore and configured to receive the light and image projection of the projector and reflect the projection onto the inner surface of the MRI machine bore to form an image.

2. The MRI-compatible projector system of claim 1, wherein the projector is a digital light processing (DLP) projector.

3. The MRI-compatible projector system of claim 1, wherein the projector is a liquid crystal on silicon (LCoS) projector.

4. The MRI-compatible projector system of claim 1, wherein the projector is a laser projector.

5. The MRI-compatible projector system of claim 1, wherein at least one of the plurality of secondary optics comprises an aspherical lens.

6. The MRI-compatible projector system of claim 1, wherein at least one of the plurality of secondary optics comprises an achromatic lens.

7. The MRI-compatible projector system of claim 1, wherein at least one of the plurality of secondary optics comprises a doublet lens.

8. The MRI-compatible projector system of claim 1, wherein at least one of the plurality of secondary optics comprises a plano-convex lens.

9. The MRI-compatible projector system of claim 1, wherein the focal length of the projector is adjustable.

10. The MRI-compatible projector system of claim 1, wherein at least one of the plurality of secondary optics is configured to perform beam collimation.

11. The MRI-compatible projector system of claim 10, wherein the secondary optic that is configured to perform beam collimation is adjustable.

12. The MRI-compatible projector system of claim 1, the system further comprising a sensor configured to detect the position of a patient table within the MRI machine bore and transfer data related to the patient table location to the projector.

13. The MRI-compatible projector system of claim 12, wherein the focal length of the projector is adjusted automatically according to the position of the patient table within the MRI machine bore.

14. The MRI-compatible projector system of claim 12, wherein the beam collimating secondary optic is adjusted automatically according to the position of the patient table within the MRI machine bore.

15. The MRI-compatible projection system of claim 1, wherein the distance between the projector and the FML is between five and nine feet.

16. The MRI-compatible projection system of claim 15, wherein the distance between the projector and the FML is approximately seven feet.

17. An MRI-compatible projector system comprising:
an RF box enclosing a projector, the projector comprising a lens and configured to output a light and image projection; and
a cylindrical waveguide configured to be in mechanical communication with the projector lens, the waveguide also comprising a plurality of secondary optics,
wherein at least one optic of the plurality of secondary optics comprises a fisheye lens configured to project the light and image projection onto the inner surface of an MRI machine bore.

18. A method for projecting an image onto an inner surface within an MRI machine bore, the method comprising the steps of:
providing a projection system comprising:
an RF enclosure enclosing a projector, the projector comprising a lens and configured to output a light and image projection;
a cylindrical waveguide configured to be in mechanical communication with the projector lens, the waveguide also comprising a plurality of secondary optics; and
a fold-minor lens (FML) disposed within an MR machine bore and configured to receive the light and image projection of the projector and reflect the projection onto the inner surface of the MRI machine bore to form an image;
placing the projector at least five feet away from the MRI machine bore;
placing the FML within the MRI machine bore;
projecting the light and image projection from the projector through the waveguide and into the FML; and
reflecting the light and image projection from the FML onto the inner surface of the MRI machine bore.

19. The method of claim 18, wherein the projecting step further comprises the step of:
adjusting the focal length of the projector as a patient table within the MRI machine bore moves.

20. The method of claim 19, wherein the projection system further comprises a sensor configured to detect the position of the patient table within the MRI machine bore and the adjusting step is accomplished automatically.
